(19) United States
(12) Patent Application Publication
(10) Pub. No.: US 2021/0228208 A1
(43) Pub. Date: Jul. 29, 2021

(54) SURGICAL STAPLING DEVICE WITH MODIFIED STAPLE FORMATION

(71) Applicant: Covidien LP, Mansfield, MA (US)

(72) Inventor: David N. Fowler, Cheshire, CT (US)

(21) Appl. No.: 16/773,315

(22) Filed: Jan. 27, 2020

(51) Int. Cl. A61B 17/072

(52) U.S. Cl. CPC A61B 17/072; A61B 2017/0042; A61B 2017/07257; A61B 2017/07271

(57) ABSTRACT

A surgical stapling device includes a tool assembly for a surgical stapling device that includes a cartridge assembly having a plurality of rows of staples positioned on opposite sides of a central knife slot and different size staples in each of the rows of staples. The tool assembly is adapted to provide a more pronounced B-shape to the distal-most staple in an inner row of staples to improve gripping of tissue.

### FIELD

This disclosure is generally related to surgical stapling devices and, more particularly, to surgical stapling devices adapted to modify staple formation at a distal end of the stapling device.

### BACKGROUND

Surgical stapling devices for stapling tissue during a surgical procedure are well known. Such devices allow a clinician to suture tissue more quickly than traditional suturing techniques to shorten the length of the surgical procedure and minimize patient trauma.

Typically, surgical stapling devices include a tool assembly having an anvil assembly and a cartridge assembly that are coupled together to facilitate movement of the tool assembly between unclamped and clamped positions. In some stapling devices, the cartridge assembly includes a staple cartridge having two or more longitudinally aligned rows of staples on each side of a central knife slot and the anvil assembly includes an anvil having a plurality of staple deforming pockets that are aligned with the staples in the staple cartridge. When the stapling device is fired, the staples are driven from the staple cartridge and are deformed in the staple deforming pockets of the anvil. The staple deforming pockets of the anvil are commonly configured to form the staples into a B-shape.

In some stapling devices, the size of the staples in the inner rows of staples nearest the central knife slot differs from the size of the staples in the outer rows of staples. Larger staples are provided in the outer rows of staples as compared to the inner row of staples to allow some blood flow within the tissue from the outer rows of staples towards the inner rows of staples to reduce any likelihood of tissue necrosis. Due to the reduced size of the staples in the inner row of staples, formation of the staples into a B-shape may be difficult to achieve. In some instances, this is undesirable.

### SUMMARY

In aspects, this disclosure generally relates to a tool assembly for a surgical stapling device that includes a cartridge assembly having a plurality of rows of staples positioned on opposite sides of a central knife slot and different size staples in each of the rows of staples. The tool assembly is adapted to provide a more pronounced B-shape to the distal-most staple in an inner row of staples to improve gripping of tissue.

One aspect of the disclosure is directed to a tool assembly including an anvil and a cartridge assembly. The cartridge assembly includes a staple cartridge and a cartridge channel. The staple cartridge is supported on the cartridge channel and defines a first central knife slot and a plurality of staple receiving slots. The staple receiving slots include three rows of staple receiving slots on each side of the first central knife slot. Each of the three rows of staple receiving slots includes an inner row of staple receiving slots positioned adjacent to the first central knife slot, an outer row of staple receiving slots, and a middle row of staple receiving slots positioned between the inner and outer rows of staple receiving slots. Each of the staple receiving slots supports a staple. The anvil is coupled to the cartridge assembly such that the tool assembly is movable between an open position and a clamped position. The anvil includes a staple deforming surface that defines a plurality of staple deforming pockets and a second central knife slot. The staple deforming pockets are aligned in three rows of staple deforming pockets on each side of the second central knife slot. Each of the three rows of staple deforming pockets includes a an inner row of staple deforming pockets positioned adjacent the second central knife slot, an outer row of staple deforming pockets, and a middle row of staple deforming pockets positioned between the inner and outer rows of staple deforming pockets. Each of the inner rows of staple deforming pockets includes a distal-most staple deforming pocket. The distal-most staple deforming pocket of the inner rows of staple deforming pockets has a configuration that is different from the configuration of the remaining staple deforming pockets in the inner rows of staple deforming pockets.

In aspects of the disclosure, each of the staple deforming pockets in the inner rows of staple deforming pockets includes spaced buckets that are defined by curved inner walls, wherein the spaced buckets are configured to form a staple aligned with the staple deforming pocket into a B-shape.

In some aspects of the disclosure, each of the spaced buckets of the distal-most staple deforming pocket in the inner rows of staple deforming pockets defines a first depth and the spaced buckets in the remaining staple deforming pockets of the inner rows of staple deforming pockets defines a second depth, wherein the first depth is greater than the second depth.

In certain aspects of the disclosure, the distal-most staple deforming pocket in each of the inner rows of staple deforming pockets defines a first length and the remaining staple deforming pockets in each of the inner rows of staple deforming pockets defines a second length, the first length being less than the second length.

In aspects of the disclosure, the staples in the inner rows of staple receiving slots are smaller than the staples in the middle and outer rows of staple receiving slots.

In some aspects of the disclosure, the staples in the middle rows of staple receiving slots are smaller than the staples in the outer rows of staple receiving slots.

In certain aspects of the disclosure, the staples in the inner rows of staple receiving slots are 2.0 mm staples, the staples in the middle rows of staple receiving slots are 2.5 mm staples, and the staples in the outer rows of staple receiving slots are 3.0 mm staples.

Other aspects of the disclosure are directed to a surgical stapling device including a handle assembly, an elongate body, and a tool assembly. The elongate body has a proximal end coupled to the handle assembly and a distal end. The tool assembly is supported on the distal end of the elongate body and includes a cartridge assembly and an anvil. The cartridge assembly includes a staple cartridge and a cartridge channel. The staple cartridge is supported on the cartridge channel and defines a first central knife slot and a plurality of staple receiving slots. The staple receiving slots include three rows of staple receiving slots on each side of the first central knife slot. Each of the three rows of staples includes an inner row of staple receiving slots positioned adjacent to the first central knife slot, an outer row of staple receiving slots, and a middle row of staple receiving slots positioned between the inner and outer rows of staple receiving slots. Each of the staple receiving slots supports a staple. The anvil is coupled to the cartridge assembly such that the tool assembly is movable between an open position and a clamped position. The anvil includes a staple deforming surface that defines a plurality of staple deforming pockets and a second central knife slot. The staple deforming pockets are aligned in three rows of staple deforming pockets on each side of the second central knife slot. Each of the three rows of staple deforming pockets includes an inner row of staple deforming pockets positioned adjacent to the second central knife slot, an outer row of staple deforming pockets, and a middle row of staple deforming pockets positioned between the inner and outer rows of staple deforming pockets. Each of the inner rows of staple deforming pockets includes a distal-most staple deforming pocket that has a configuration that is different from the configuration of remaining staple deforming pockets in the inner rows of staple deforming pockets. Each of the staple deforming pockets in the inner rows of staple deforming pockets includes spaced buckets defined by curved inner walls. The spaced buckets are configured to form the staple aligned with the staple deforming pocket into a B-shape.

Other aspects of the disclosure are directed to a tool assembly including an anvil, a cartridge assembly, and a plurality of staples. The anvil includes a staple deforming surface that defines a plurality of staple deforming pockets and a first central knife slot. The cartridge assembly is coupled to the anvil such that the tool assembly is movable between an open position and a clamped position. The cartridge assembly includes a staple cartridge and a cartridge channel. The staple cartridge is supported on the cartridge channel and defines a second central knife slot and a plurality of staple receiving slots. The staple receiving slots includes three rows of staple receiving slots on each side of the second central knife slot. Each of the three rows of staple receiving slots includes an inner row of staple receiving slots positioned adjacent to the first central knife slot, an outer row of staple receiving slots, and a middle row of staple receiving slots positioned between the inner and outer rows of staple receiving slots. Each of the plurality of staples is received within a respective one of the staple receiving slots. Each of the inner rows of staple receiving slots includes a distal-most staple receiving slot. The staples received in the distal-most staple receiving slots of the inner rows of staple receiving slots is larger than the staples received within remaining staple receiving slots of the inner rows of staple receiving slots.

In aspects of the disclosure, the staples received in the remaining staple receiving slots of the inner row of staple receiving slots are smaller than the staples in the middle and outer rows of staple receiving slots.

In some aspects of the disclosure, the staples received in the middle row of staple receiving slots are smaller than the staples received in the outer rows of staple receiving slots.

In certain aspects of the disclosure, the remaining staples received in the inner rows of staple receiving slots are 2.0 mm staples, the staples in the middle rows of staple receiving slots are 2.5 mm staples, and the staples in the outer rows of staple receiving slots are 3.0 mm staples

Other features of the disclosure will be appreciated from the following description.

### BRIEF DESCRIPTION OF DRAWINGS

Various aspects of the disclosure are described herein below with reference to the drawings, wherein:

FIG. 1 is a side perspective view of a surgical stapling device including a tool assembly in accordance with aspects of this disclosure;

FIG. 2 is an enlarged view of the indicated area of detail shown in FIG. 1;

FIG. 3 is a perspective from below an anvil assembly of the tool assembly shown in FIG. 2;

FIG. 4 is a cross-sectional view taken along section line 4-4 of FIG. 3;

FIG. 5 is a cross-sectional view taken along section line 5-5 of FIG. 6;

FIG. 6 is a top view of staple lines formed in tissue treated by the surgical stapling device shown in FIG. 1;

FIG. 7 is side perspective exploded view of a staple cartridge in accordance with other aspects of the disclosure;

FIG. 8 is a cross-sectional view taken along section line 8-8 of FIG. 7; and

FIG. 9 is a cross-sectional view taken along section line 9-9 of FIG. 7.

### DETAILED DESCRIPTION

The disclosed surgical stapling device will now be described in detail with reference to the drawings in which like reference numerals designate identical or corresponding elements in each of the several views. However, it is to be understood that the aspects of the disclosure are merely exemplary of the disclosure and may be embodied in various forms. Well-known functions or constructions are not described in detail to avoid obscuring the disclosure in unnecessary detail. Therefore, specific structural and functional details disclosed herein are not to be interpreted as limiting, but merely as a basis for the claims and as a representative basis for teaching one skilled in the art to variously employ the disclosure in virtually any appropriately detailed structure. In addition, directional terms such as front, rear, upper, lower, top, bottom, distal, proximal, and similar terms are used to assist in understanding the description and are not intended to limit the disclosure.

In this description, the term “proximal” is used generally to refer to that portion of the device that is closer to a clinician, while the term “distal” is used generally to refer to that portion of the device that is farther from the clinician. In addition, the term “endoscopic” is used generally used to refer to endoscopic, laparoscopic, arthroscopic, and/or any other procedure conducted through a small diameter incision or cannula. Further, the term “clinician” is used generally to refer to medical personnel including doctors, nurses, and support personnel.

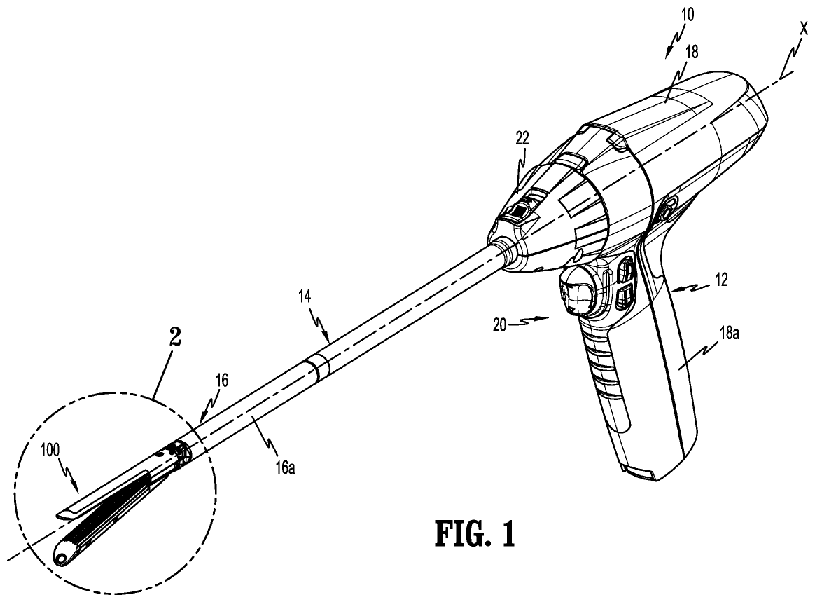

FIG. 1 illustrates the disclosed surgical stapling device shown generally as stapling device 10 which includes a handle assembly 12, an elongate body 14, and a tool assembly 100. Although not described in detail herein, the tool assembly 100 can form part of a reload assembly 16 that is releasably coupled to a distal portion of the elongate body 14 and includes a proximal body portion 16a and the tool assembly 100. Alternately, the tool assembly 100 can be fixedly secured to a distal portion of the elongate body 14.

The elongate body 14 defines a longitudinal axis “X”. The handle assembly 12 is coupled to a proximal end of the elongate body 14 and includes a body 18 that defines hand grip 18a, a plurality of actuator buttons 20, and a rotation knob 22. The rotation knob 22 is rotatably supported on a distal portion of the handle assembly 12 and supports the elongate body 14 to facilitate rotation of the elongate body 14 and the tool assembly 100 about the longitudinal axis “X” in relation to the handle assembly 12. The actuator buttons 20 control operation of the various functions of the stapling device 10 including approximation, firing and cutting. Although the stapling device 10 is illustrated as an electrically powered stapling device, it is envisioned that the disclosed tool assembly 100 would also be suitable for use with a manually powered surgical stapling device. U.S. Pat. No. 9,055,943 (the '943 patent) discloses a surgical stapling device including a powered handle assembly and U.S. Pat. No. 6,241,139 (the '139 patent) discloses a manually actuated handle assembly. For a more detailed description of exemplary handle assemblies of the stapling device 10, see, e.g., the '943 and '139 patents.

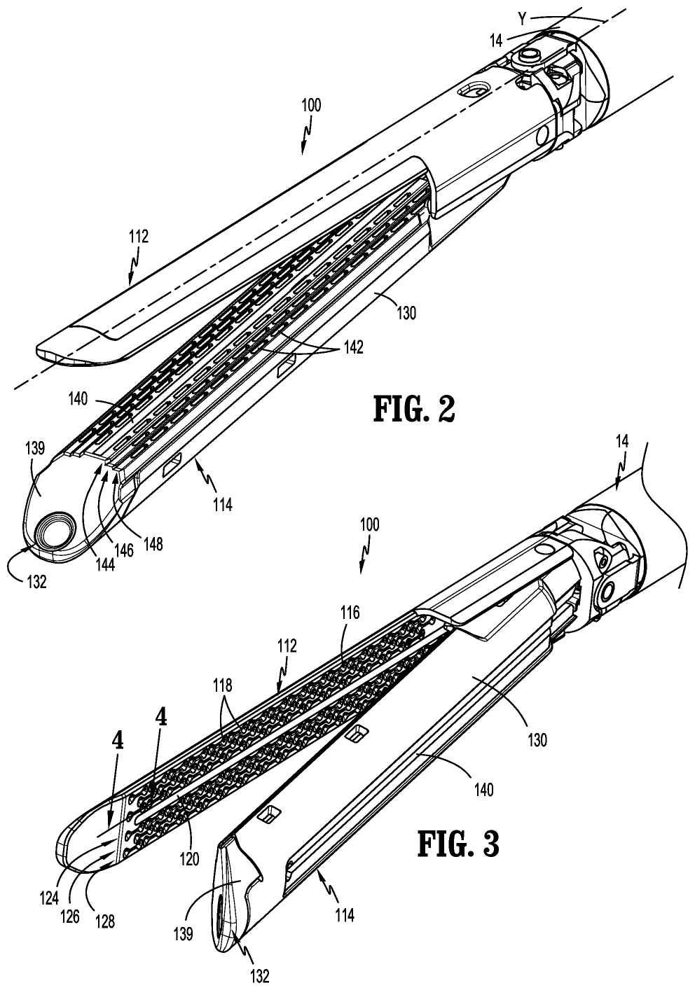

FIGS. 2 and 3 illustrate the tool assembly 100 of the stapling device 10. The tool assembly 100 defines a longitudinal axis “Y” (FIG. 2) and includes an anvil assembly 112 and a cartridge assembly 114. The anvil assembly 112 and the cartridge assembly 114 are movable in relation to each other between an open or unclamped position (FIG. 2) and a clamped position. The anvil assembly 112 includes a staple deforming surface 116 (FIG. 3) that defines a plurality of staple deforming pockets 118 (FIG. 3) and a central knife slot 120. The staple deforming pockets 118 are aligned in three rows on each side of the central knife slot 120 including an inner row 124 positioned adjacent to the central knife slot 120, a middle row 126, and an outer row 128.

The cartridge assembly 114 (FIG. 2) includes a cartridge channel 130 and a staple cartridge 132. The cartridge channel 130 receives and supports the staple cartridge 132. In aspects of the disclosure, the staple cartridge 132 is removable from the cartridge channel 130 to facilitate reuse of the stapling device 10. Alternately, the staple cartridge 132 is fixedly secured within the cartridge channel 130 and the reload assembly 16 (FIG. 1) can be removed from the stapling device 10 to facilitate reuse of the stapling device 10.

The staple cartridge 132 includes a cartridge body 139 that defines a central knife slot 140 and a plurality of staple receiving pockets 142. The staple receiving pockets 142 are aligned in three rows on each side of the central knife slot 140 including an inner row 144 positioned adjacent to the central knife slot 140 (FIG. 2), a middle row 146, and an outer row 148. Each of the staple receiving pockets 142 supports a staple 150 (FIG. 5) such that the staples 150 are aligned in three rows on each side of the central knife slot 140 within the staple cartridge 132. When the tool assembly 100 is in a clamped position, the plurality of staple receiving pockets 142 in the staple cartridge 132 are aligned with the staple deforming pockets 118 in the anvil assembly 112 such that each of the staples 150 ejected from the staple cartridge 132 is deformed within a respective staple deforming pocket 118 of the anvil assembly 112.

In aspects of the disclosure, the staples 150 in the inner, middle, and outer rows 144, 146, and 148 respectively, of staple receiving slots 142 are different sizes. In some aspects of the disclosure, the staples in the inner rows 144 are 2.0 mm staples, the staples in the middle rows 146 are 2.5 mm staples, and the staples in the outer rows 148 are 3.0 mm staples. It is envisioned that the size of the staples 150 in the different rows of staples can vary will be selected based upon the intended use of the stapling device. A variety of different size staples are envisioned. Larger staples are provided in the outer rows 148 of staples as compared to the inner rows 144 of staples to allow some blood flow within the tissue from the outer rows 148 of staples 150 towards the inner rows 144 of staples 150 to reduce any likelihood of tissue necrosis along the staple line when the staples 50 are formed in tissue “T”.

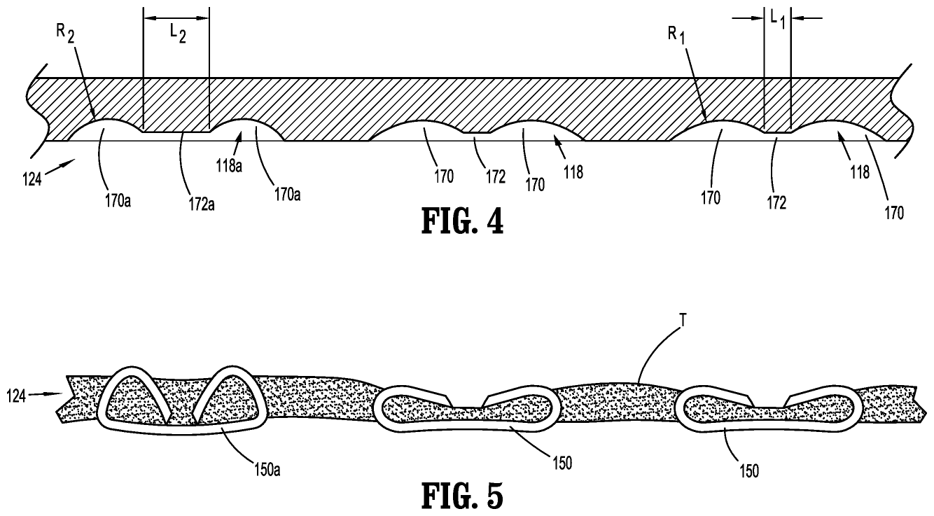

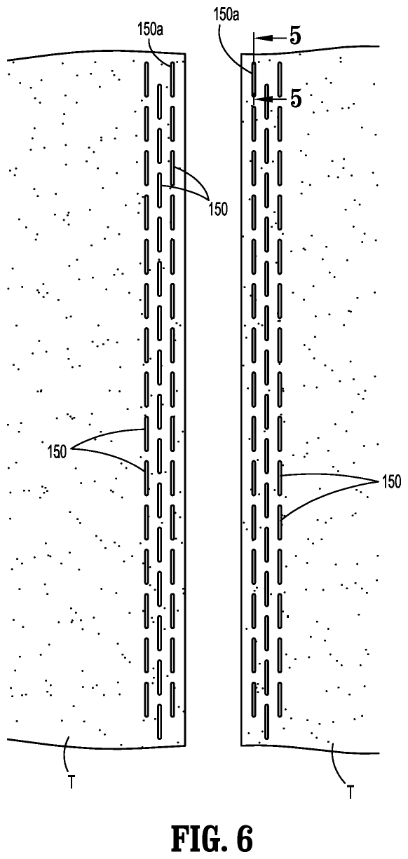

FIG. 4 illustrates a cross-sectional view taken through a distal end portion of the staple deforming surface 116 of the anvil assembly 112 illustrating the distal-most staple deforming pocket 118a in the inner row 124 of staple deforming pockets 118. As illustrated, each of the staple deforming pockets 118 includes spaced buckets 170 that are interconnected by a linear channel 172. The spaced buckets 170 are defined by curved inner walls that are configured to deform the staples 150 into a B-shaped configuration. In aspects of the disclosure, the configuration of the distal-most staple deforming pocket 118a differs from the configuration of other staple deforming pockets 118 in the inner row 124 of staple deforming pockets 118. More specifically, although the depth and the length of the distal-most staple deforming pocket 118a and the proximally located staple deforming pockets 118 are the same, the radius of curvatures “R1” and “R2” defining the spaced buckets 170a and 170 in the staple deforming pockets 118 and 118a, respectively, are different. Since the radius of curvatures “R1” and R2″ defining the buckets 170 and 170a are different and the overall lengths of the staple deforming pockets 118 and 118a remain the same, the lengths “L1” and “L2” of the linear channel 172 and 172a in the staple deforming pockets 118 and 118a, respectively, are also different. In aspects of the disclosure, “R1” is from about 0.043 inches to about 0.047 inches and “R2” is from about 0.034 inches to about 0.025 inches, whereas “L1” is from about 0.025 inches to about 0.033 inches and “L2” is from about 0.035 inches to about 0.043 inches. The configuration of the distal-most staple deforming pocket 118a as compared to the configuration of the proximally located pockets 118 deforms the distal-most staple 150a of the inner rows 124 of staples 150 into a more pronounced B-shape to provide a deformed staple 150a that has better gripping capability in tissue “T” (FIG. 6) than the staples 150 formed in the staple-pockets 118 located proximally of the distal-most staple 150a.

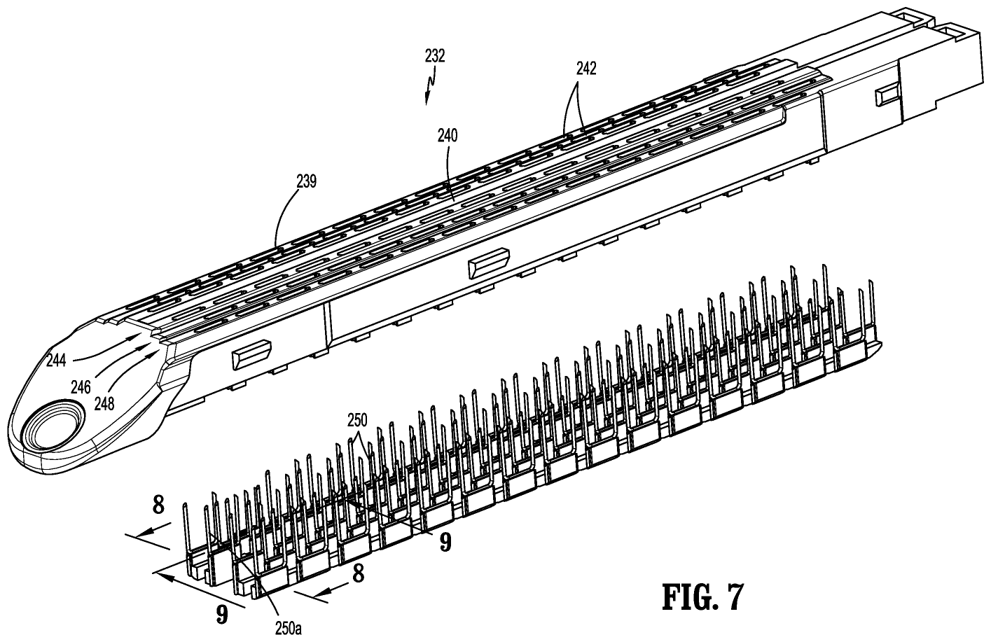

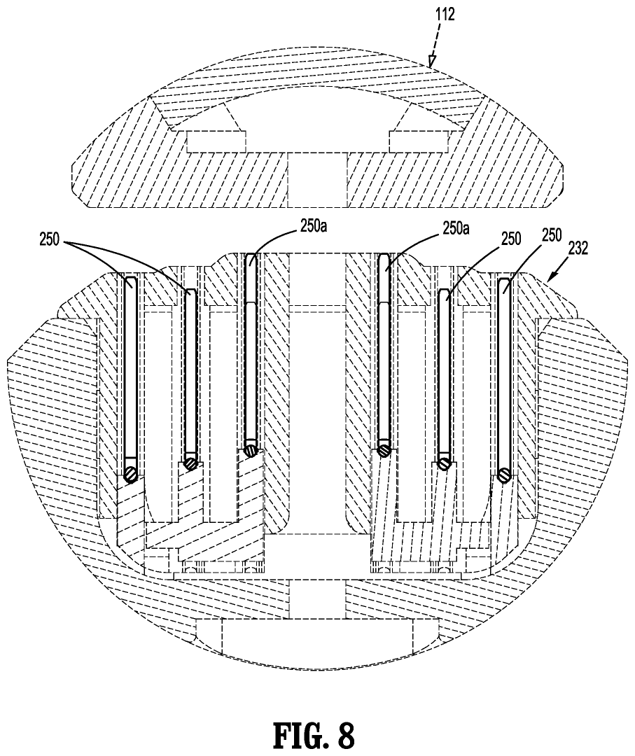

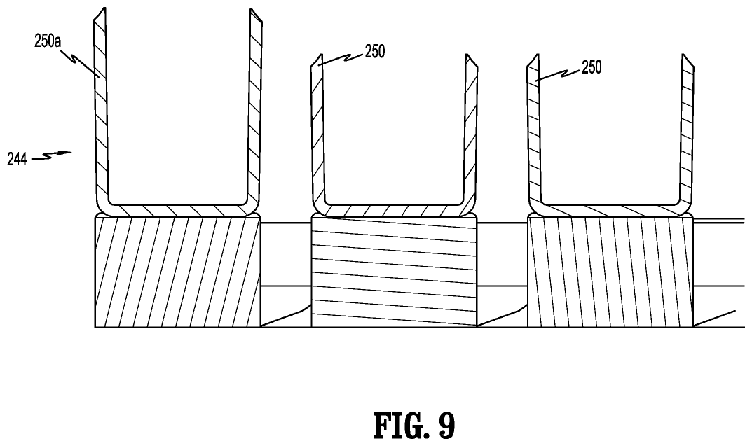

FIGS. 7-9 illustrate alternative aspects of the disclosure including cartridge assembly 214 (FIG. 8) including a staple cartridge 232 that is adapted to provide a distal-most staple 250a in an inner row 244 of staples 250 with a more pronounced B-shape. The staple cartridge 232, as described above in regard to staple cartridge 132 (FIG. 2), includes a cartridge body 239 that defines a central knife slot 240 and a plurality of staple receiving slots 242. The staple receiving slots 242 are aligned in three rows on each side of the central knife slot 240 including an inner row 244, a middle row 246, and an outer row 248. Each of the staple receiving slots 242 supports a staple 250. When the tool assembly 100 (FIG. 1) is in a clamped position, the plurality of staple receiving slots 242 in the staple cartridge 232 are aligned with the staple deforming pockets 118 (FIG. 2) in the anvil assembly 112 (FIG. 2) such that staples 250 ejected from the staple cartridge 232 are deformed within respective staple deforming pockets 118 (FIG. 2) of the anvil assembly 112.

In aspects of the disclosure, the different rows of staple receiving pockets 242 of the staple cartridge 232 support three different size staples. For example, the staples 250 in the inner rows 244 of staple receiving pockets 242 may be 2.0 mm staples, the staples 250 in the middle rows 246 of staple receiving pockets 242 may be 2.5 mm staples, and the staples 250 in the outer rows 248 of staple receiving pockets 242 may be 3.0 mm staples. It is envisioned that the size of the staples 250 in the different rows of staples 250 may vary and will be selected based upon the intended use of the stapling device.

In this aspect of the disclosure, the size of the distal-most staple 250a in the inner rows 244 of staple receiving slots 242 is larger than the remaining staples 250 in the inner rows 244 of staple receiving slots 242. For example, the staples 250 proximal to the distal-most staple 250a in the inner rows 244 of staple receiving slots 242 may be 2.0 mm staples 250 and the distal-most staple 250a may be a 2.5 mm staple or a 3.0 mm staple 250a. The increased length of the distal-most staple 250a allows the staple 250 to form a more pronounced B-shape to improve the tissue gripping capability of the distal-most staple 250a in the inner rows 244 of staple pockets 244. In this aspect of the disclosure, the staple deforming pockets 118 in the anvil assembly 12 are uniform along the length of the inner rows 124 (FIG. 3) of staple deforming pockets 118, i.e., the distal-most staple deforming pocket 242 has the same configuration as the proximally located staple deforming pockets 242 in the inner rows 244 of staple deforming pockets 242.

Persons skilled in the art will understand that the devices and methods specifically described herein and illustrated in the accompanying drawings are non-limiting exemplary aspects of the disclosure. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described aspects of the disclosure. Accordingly, the disclosure is not to be limited by what has been particularly shown and described, except as indicated by the appended claims.

What is claimed is:

1. A tool assembly comprising:
a cartridge assembly including a staple cartridge and a cartridge channel, the staple cartridge supported on the cartridge channel and defining a first central knife slot and a plurality of staple receiving slots, the staple receiving slots including three rows of staple receiving slots on each side of the first central knife slot, each of the three rows of staple receiving slots including an inner row of staple receiving slots positioned adjacent to the first central knife slot, an outer row of staple receiving slots, and a middle row of staple receiving slots positioned between the inner and outer rows of staple receiving slots, each of the staple receiving slots supporting a staple; and
an anvil coupled to the cartridge assembly such that the tool assembly is movable between an open position and a clamped position, the anvil including a staple deforming surface defining a plurality of staple deforming pockets and a second central knife slot, the staple deforming pockets being aligned in three rows of staple deforming pockets on each side of the second central knife slot, each of the three rows of staple deforming pockets including a an inner row of staple deforming pockets positioned adjacent the second central knife slot, an outer row of staple deforming pockets, and a middle row of staple deforming pockets positioned between the inner and outer rows of staple deforming pockets, wherein each of the inner rows of staple deforming pockets includes a distal-most staple deforming pocket, the distal-most staple deforming pocket of the inner rows of staple deforming pockets having a configuration different from the configuration of remaining staple deforming pockets in the inner rows of staple deforming pockets.

2. The tool assembly of claim 1, wherein each of the staple deforming pockets in the inner rows of staple deforming pockets includes spaced buckets defined by curved inner walls, the spaced buckets being configured to form the staple aligned with the staple deforming pocket into a B-shape.

3. The tool assembly of claim 2, wherein each of the spaced buckets of the distal-most staple deforming pocket in the inner rows of staple deforming pockets is defined by a radius of curvature “R2” and each of the spaced buckets in the remaining staple deforming pockets of the inner rows of staple deforming pockets are defined by a second radius of curvature “R1”, wherein “R1” is greater than “R2”.

4. The tool assembly of claim 3, wherein “R1” is from about 0.043 inches to about 0.047 inches and “R2” is from about 0.034 inches to about 0.025 inches.

5. The tool assembly of claim 4, wherein the staples in the inner rows of staple receiving slots are smaller than the staples in the middle and outer rows of staple receiving slots.

6. The tool assembly of claim 5, wherein the staples in the middle rows of staple receiving slots are smaller than the staples in the outer rows of staple receiving slots.

7. The tool assembly of claim 6, wherein the staples in the inner rows of staple receiving slots are 2.0 mm staples, the staples in the middle rows of staple receiving slots are 2.5 mm staples, and the staples in the outer rows of staple receiving slots are 3.0 mm staples.

8. A surgical stapling device comprising:
a handle assembly;
an elongate body having a proximal end and a distal end, the proximal end coupled to the handle assembly;
a tool assembly supported on the distal end of the elongate body, the tool assembly including:
a cartridge assembly including a staple cartridge and a cartridge channel, the staple cartridge supported on the cartridge channel and defining a first central knife slot and a plurality of staple receiving slots, the staple receiving slots including three rows of staple receiving slots on each side of the first central knife slot, each of the three rows of staple receiving slots including an inner row of staple receiving slots positioned adjacent to the first central knife slot, an outer row of staple receiving slots, and a middle row of staple receiving slots positioned between the inner and outer rows of staple receiving slots, each of the staple receiving slots supporting a staple; and
an anvil coupled to the cartridge assembly such that the tool assembly is movable between an open position and a clamped position, the anvil including a staple deforming surface defining a plurality of staple deforming pockets and a second central knife slot, the staple deforming pockets being aligned in three rows of staple deforming pockets on each side of the second central knife slot, each of the three rows of staple deforming pockets including an inner row of staple deforming pockets positioned adjacent the second central knife slot, an outer row of staple deforming pockets, and a middle row of staple deforming pockets positioned between the inner and outer rows of staple deforming pockets, wherein each of the inner rows of staple deforming pockets includes a distal-most staple deforming pocket, the distal-most staple pocket of the inner rows of staple pockets having a configuration different from the configuration of the remaining staple deforming pockets in the inner rows of staple deforming pockets.

9. The surgical stapling device of claim 8, wherein each of the staple deforming pockets in the inner rows of staple deforming pockets includes spaced buckets defined by curved inner walls, the spaced buckets being configured to form the staple aligned with the staple deforming pocket into a B-shape.

10. The surgical stapling device of claim 9, wherein each of the spaced buckets of the distal-most staple deforming pocket in the inner rows of staple deforming pockets is defined by a radius of curvature “R2” and each of the spaced buckets in the remaining staple deforming pockets of the inner rows of staple deforming pockets are defined by a second radius of curvature “R1”, wherein “R1” is greater than “R2”.

11. The surgical stapling device of claim 10, wherein “R1” is from about 0.043 inches to about 0.047 inches and “R2” is from about 0.034 inches to about 0.025 inches.

12. The surgical stapling device of claim 11, wherein the staples in the inner rows of staple receiving slots are smaller than the staples in the middle and outer rows of staple receiving slots.

13. The surgical stapling device of claim 12, wherein the staples in the middle rows of staple receiving slots are smaller than the staples in the outer rows of staple receiving slots.

14. The surgical stapling device of claim 13, wherein the staples in the inner rows of staple receiving slots are 2.0 mm staples, the staples in the middle rows of staple receiving slots are 2.5 mm staples, and the staples in the outer rows of staple receiving slots are 3.0 mm staples.

15. A tool assembly comprising:
an anvil including a staple deforming surface defining a plurality of staple deforming pockets and a first central knife slot;
a cartridge assembly coupled to the anvil such that the tool assembly is movable between an open position and a clamped position, the cartridge assembly including a staple cartridge and a cartridge channel, the staple cartridge supported on the cartridge channel and defining a second central knife slot and a plurality of staple receiving slots, the staple receiving slots including three rows of staple receiving slots on each side of the second central knife slot, each of the three rows of staple receiving slots including an inner row of staple receiving slots positioned adjacent to the first central knife slot, an outer row of staple receiving slots, and a middle row of staple receiving slots positioned between the inner and outer rows of staple receiving slots; and
a plurality of staples, each of the plurality of staples being received within a respective one of the staple receiving slots, wherein each of the inner rows of staple receiving slots includes a distal-most staple receiving slot, and the staples received in the distal-most staple receiving slots of the inner rows of staple receiving slots is larger than the staples received within remaining staple receiving slots of the inner rows of staple receiving slots.

15. The tool assembly of claim 14, wherein the staples received in the remaining staple receiving slots of the inner row of staple receiving slots are smaller than the staples in the middle and outer rows of staple receiving slots.

16. The tool assembly of claim 15, wherein the staples received in the middle row of staple receiving slots are smaller than the staples received in the outer rows of staple receiving slots.

17. The surgical stapling device of claim 16, wherein the remaining staples received in the inner rows of staple receiving slots are 2.0 mm staples, the staples in the middle rows of staple receiving slots are 2.5 mm staples, and the staples in the outer rows of staple receiving slots are 3.0 mm staples.
